(19) United States
(12) Patent Application Publication
(10) Pub. No.: US 2017/0151085 A1
(43) Pub. Date: Jun. 1, 2017

(54) EARHOOK ANTI-SNORING DEVICE AND ANTI-SNORING SYSTEM

(71) Applicants: Inventec (Pudong) Technology Corporation, Shanghai (CN); INVENTEC CORPORATION, Taipei City (TW)

(72) Inventors: Yung-Huang CHANG, TAIPEI CITY (TW); Chih-Hao LU, TAIPEI CITY (TW); An-Tai LEE, TAIPEI CITY (TW); Chun-Chi LIN, TAIPEI CITY (TW); Chih-Ching WANG, TAIPEI CITY (TW)

(21) Appl. No.: 15/092,627

(22) Filed: Apr. 7, 2016

(30) Foreign Application Priority Data
Nov. 27, 2015 (CN) 201510851622.X

(51) Int. Cl. A61F 5/56; H04R 1/10

(52) U.S. Cl. CPC A61F 5/56; H04R 1/1091; H04R 1/105

(57) ABSTRACT

An earhook anti-snoring device is disclosed. The earhook anti-snoring device includes a casing, a base plate, a control unit, a sound-receiving device, a sound-generating device and a vibrating device. The base plate is arranged in the casing, and the control unit and the sound-receiving device are positioned on a side of the base plate. The position of the sound-receiving device corresponds to a hollow pipe arranged on the casing. The sound-generating device and the vibrating device are positioned on another side of the base plate and the sound-generating device is opposite to the sound-receiving device. The position of the sound-generating device corresponds to another hollow pipe arranged on the casing, and the sound-generating device is directed to an ear canal of a user along the hollow pipe.

### RELATED APPLICATIONS

This application claims priority to Chinese Application Serial Number 201510851622.X, filed Nov. 27, 2015, which is herein incorporated by reference.

### BACKGROUND

Field of Invention

The disclosure relates to an earhook anti-snoring device and an anti-snoring system, and in particular to an earhook anti-snoring device and an anti-snoring system which stop snoring by triggering a sound signal and a vibrating signal.

Description of Related Art

Good sleep quality is favorable for physical health. However, due to various external factors, modern people cannot have good sleep quality, and thus the working performance and physical health are affected. One of these external factors affecting the sleep quality is snoring. Due to their constitutions or other reasons, a small number of people may snore during sleep, which not only affects their sleep quality, but also possibly affects others. As a result, anti-snoring devices arise. At present, most of existing anti-snoring devices are assembled on the hand. However, since snores come from the mouth and the nose, such devices cannot collect the snores accurately, even only collect external background sounds, and thus cannot achieve an anti-snoring effect accurately.

### SUMMARY

An implementation aspect of the disclosure provides an earhook anti-snoring device which includes a casing, a base plate, a control unit, a sound-receiving device, a sound-generating device and a vibrating device. The base plate is arranged in the casing, and the control unit and the sound-receiving device are positioned on a side of the base plate. The position of the sound-receiving device corresponds to a hollow pipe arranged on the casing. The sound-generating device and the vibrating device are positioned on another side of the base plate and the sound-generating device is opposite to the sound-receiving device. The position of the sound-generating device corresponds to another hollow pipe arranged on the casing, and the sound-generating device is directed to an ear canal of a user along the hollow pipe.

Another implementation aspect of the disclosure provides an anti-snoring system which includes an earhook anti-snoring device and a charging base. The charging base includes a spring needle connector and a magnet; and the spring needle connector includes two spring needles. The magnet is adjacent to the spring needle connector and is located adjacently between the two spring needles.

### BRIEF DESCRIPTION OF THE DRAWINGS

FIG. 1 illustrates a schematic view of an anti-snoring system according to an embodiment of disclosure;

FIG. 2 illustrates a schematic view of an earhook anti-snoring device according to an embodiment of disclosure;

FIG. 3 illustrates an internal top view of an earhook anti-snoring device according to an embodiment of disclosure;

FIG. 4 illustrates an internal bottom view of an earhook anti-snoring device according to an embodiment of disclosure;

FIG. 5 illustrates a schematic side sectional view of the earhook anti-snoring device in FIG. 4; and [FIG. 6 illustrates a schematic view of a charging base according to an embodiment of disclosure.

### DETAILED DESCRIPTION

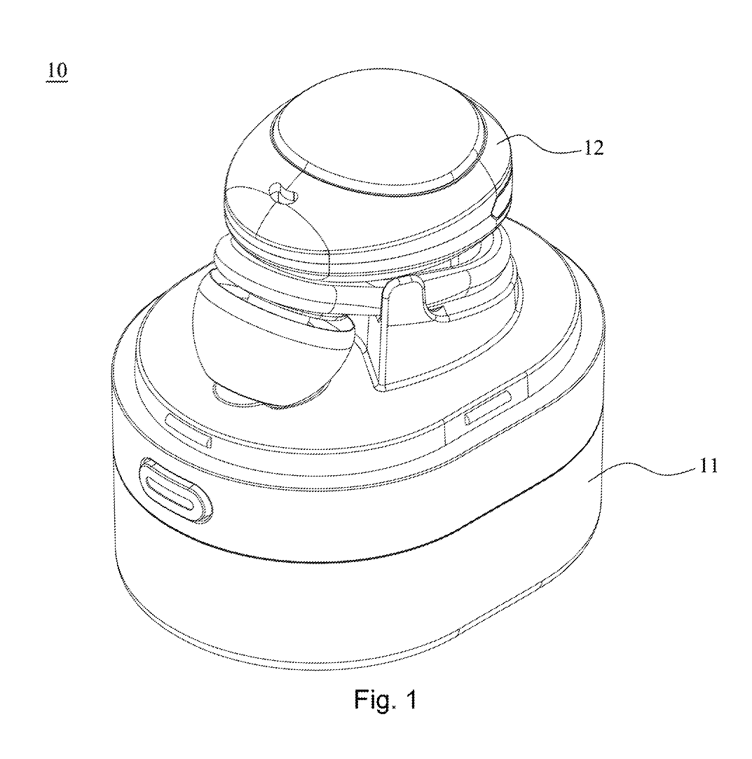

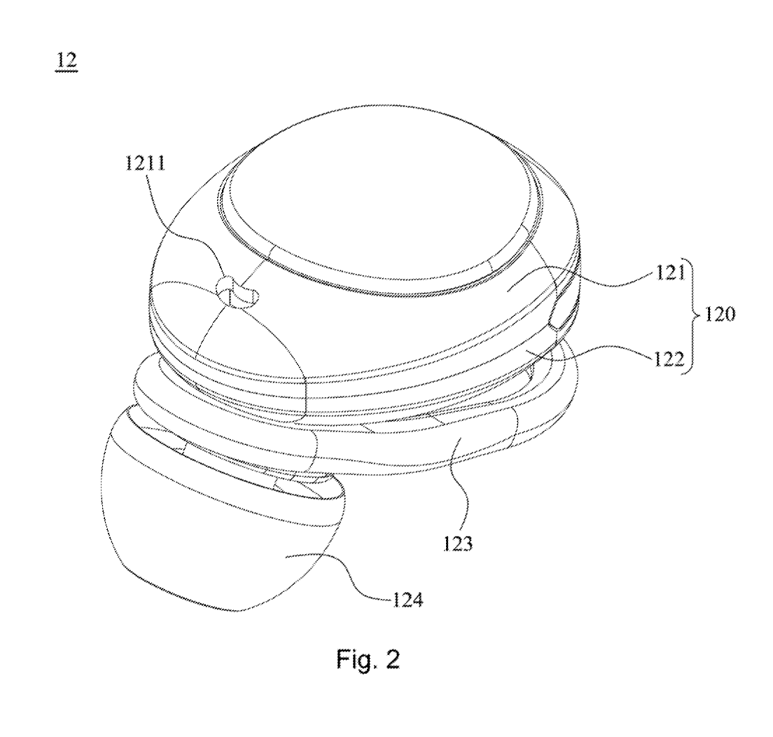

FIG. 1 illustrates a schematic view of an anti-snoring system 10 according to an embodiment of disclosure. As shown in FIG. 1, the anti-snoring system 10 includes a charging base 11 and an earhook anti-snoring device 12. Also referring to FIG. 2, it illustrates a schematic view of the earhook anti-snoring device 12 according to an embodiment of disclosure. As shown in FIG. 2, the earhook anti-snoring device 12 includes a casing 120, a ear hook 123 and an earplug 124, wherein the ear hook 123 is connected with the casing 120 and the earplug 124 and used for enabling a user to suspend and fix the earhook anti-snoring device 12 to the ear. The casing 120 includes a top casing 121 and a bottom casing 122. The top casing 121 is provided with a hollow pipe 1211, and the bottom casing 122 is provided with a hollow pipe 1221.

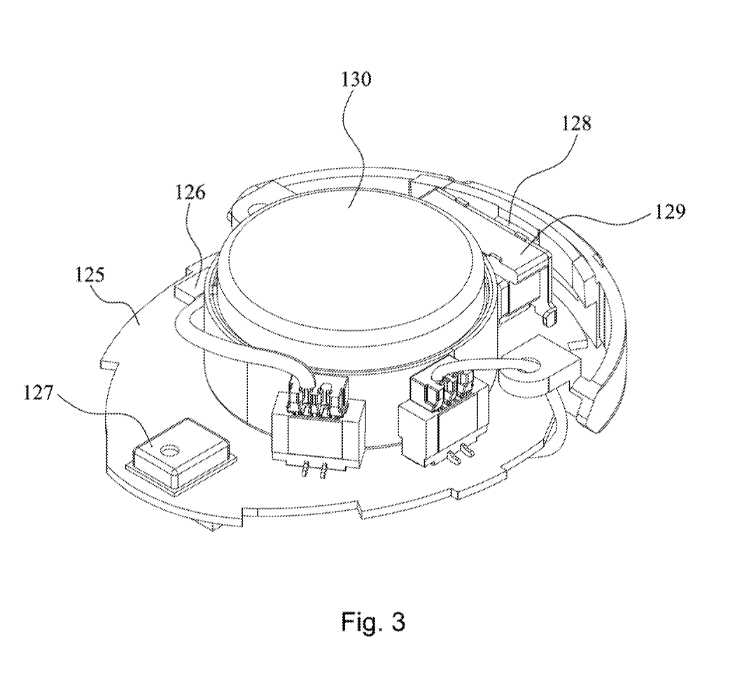

Also referring to FIG. 3, it illustrates an internal top view of an earhook anti-snoring device 12 according to an embodiment of disclosure. As shown in FIG. 3, the earhook anti-snoring device 12 internally includes a base plate 125. A control unit 126, a sound-receiving device 127, a button 128, a switch unit 129 and a battery 130 are arranged on a side of the base plate 125, wherein the button 128 is adjacent to the switch unit 129, and the battery 130 is electrically connected with the base plate 125 through a line and used for supplying power to the earhook anti-snoring device 12.

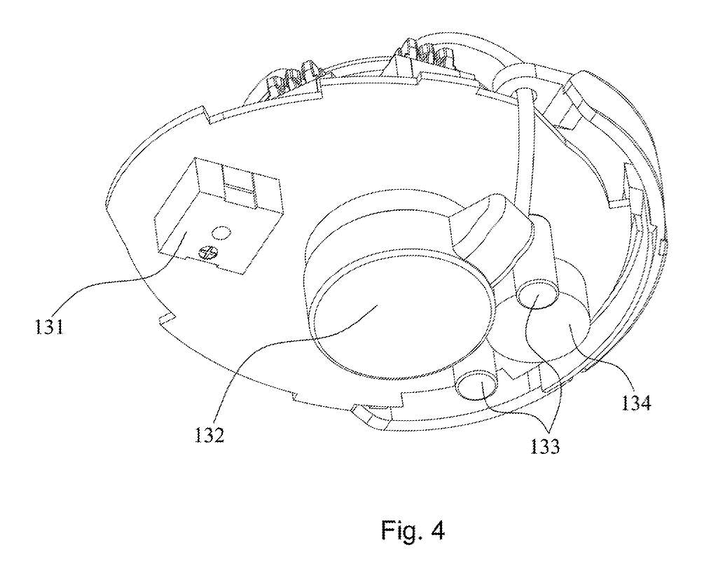

Also referring to FIG. 4, it illustrates an internal bottom view of an earhook anti-snoring device 12 according to an embodiment of disclosure. As shown in FIG. 4, a sound-generating device 131, a vibrating device 132, a spring connector 133 and a magnet 134 are arranged on another side of the base plate 125, wherein the magnet 134 is positioned between two spring contacts close to the spring connector 133, and the magnet 134 is used for placing the earhook anti-snoring device 12 above the charging base 11 accurately.

The sound-receiving device 127 corresponds to the hollow pipe 1211 arranged on the top casing 121 of FIG. 1, and the sound-receiving device 127 is used for receiving sound data coming from an external user through the hollow pipe 1211.

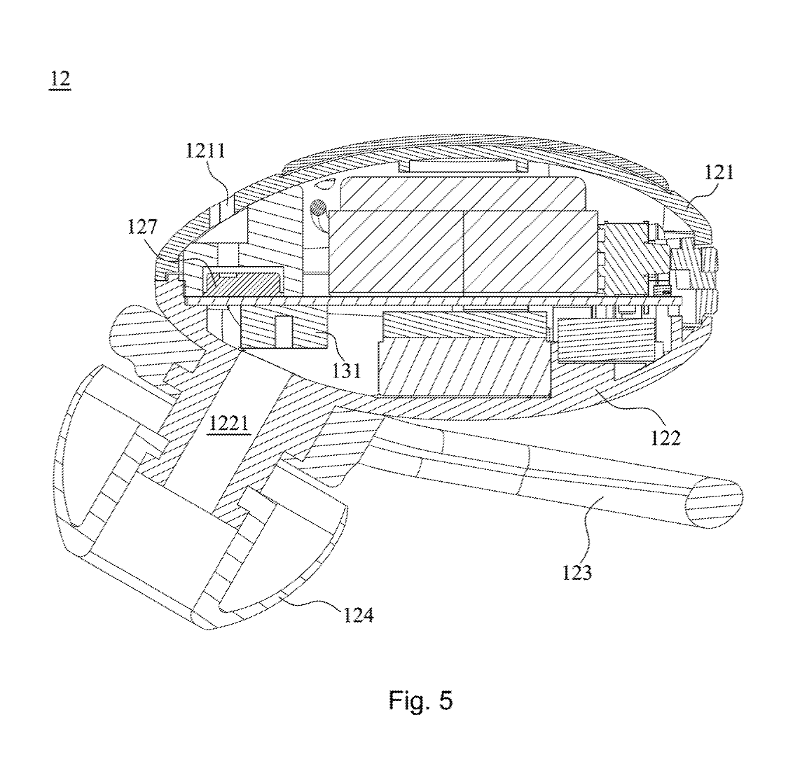

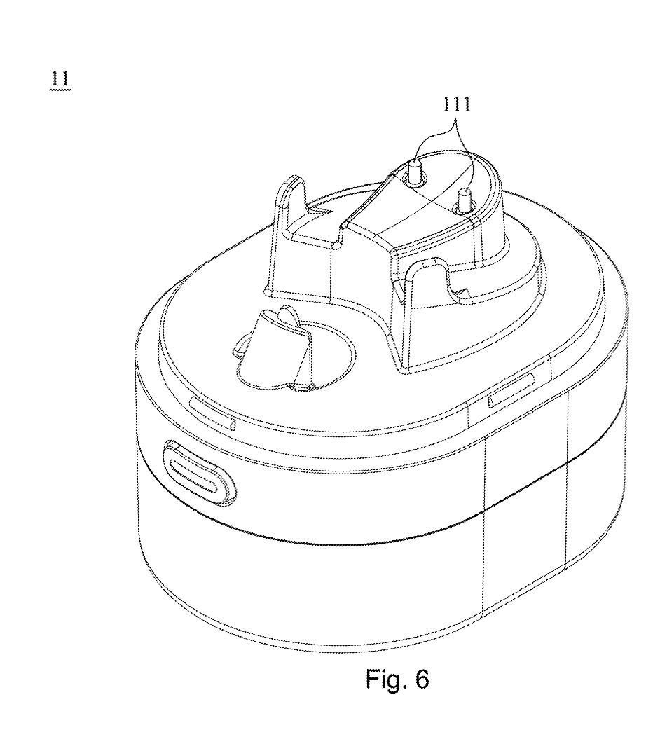

Also referring to FIG. 5, it illustrates a schematic side sectional view of the earhook anti-snoring device 12 in FIG. 4. As shown in FIG. 5, the sound-generating device 131 corresponds to the hollow pipe 1221 arranged on the bottom easing 122, and is directed to an ear canal of a user along the hollow pipe 1221. The sound-generating device 131 is used for generating a sound signal, and when the sound-generating device 131 generates the sound signal, the sound signal is transmitted through the earplug 124 along the hollow pipe 1221 to the ear canal of the user. The vibrating device 132 is used for generating a vibrating signal. The control unit 126 is used for triggering the sound-generating device 131 and the vibrating device 132 according to the received sound data.

At present, the anti-snoring mechanism of existing anti-snoring devices of other types (such as a bracelet type anti-snoring device or a neck-worn type anti-snoring device) may include generating sounds. However, for generating sound at the bracelet or the neck, a high sound volume is required for getting attention from the user, and others nearby may also be affected. The earhook anti-snoring device 12 of the disclosure is hung on the ear of the user, and the sound-generating device 131 may transmit a sound signal into the ear canal of the user through the hollow pipe 1221 arranged on the bottom casing 122. Therefore, it can be detected by the user more easily when the sound-generating device 131 sends out a sound signal. Besides, as the position where the sound is generated is in the ear canal of the user and is separated from the outside, others are not liable to be affected. Similarly, the vibrating signal generated by the vibrating device 132 is close to the ear canal of the user and may be detected by the user more easily.

When the user wears the ear hook 123 of the earhook anti-snoring device 12 before sleep and places the earplug 124 in the ear canal,the button 128 is pressed to start the switch unit 129 of the earhook anti-snoring device 12, and the sound-receiving device 127 starts to receive external sound data through the hollow pipe 1211 arranged on the top casing 121.

At present, the sound-receiving positions of existing anti-snoring devices of other types (such as a bracelet type anti-snoring device or a neck-worn type anti-snoring device) are far from sound-generating, parts of a user, such as the mouth and the nose, and thus it is difficult to detect snores generated by the user. The earhook anti-snoring device 12 of the disclosure is hung on the ear of the user, and in comparison the earhook anti-snoring device 12 is close to the sound-generating parts of the user, such as the mouth and the nose. Moreover, the sound-receiving device 127 receives sounds outwards through the hollow pipe 1211 arranged on the top casing 121 and is not easily blocked by an auricle of the user, thereby receiving the sounds generated by the user conveniently. The earhook anti-snoring device 12 of the disclosure can receive the sound data of the user himself/herself (such as snores). However, under some conditions, when the environmental background sounds (such as sounds generated by home appliances such as an electric fan and an air conditioner) are obvious, possibly the environmental background sounds are also received by the sound-receiving device 127.

Since the disclosure mainly disclosures the earhook anti-snoring device 12, the control unit 126 makes a judgment according to sound data collected by the sound-receiving device 127, and when it is judged that the sound data is snores of the user, the control unit 126 triggers the sound-generating device 131 and the vibrating device 132.

When the control unit 126 triggers the sound-generating device 131, a sound signal generated by the sound-generating device 131 passes through the hollow pipe 1221 arranged on the bottom casing 122 and is transmitted to the ear canal of the user through the hollow pipe 1221. In some embodiments, the frequency of the sound signal generated by the sound-generating device 131 is 300 HZ, 350 HZ, 500 HZ or 1 KHZ. The duration of the sound signal is 1 s, 1.5 s or 3 s. On the other hand, when the control unit 126 triggers the vibrating device 132, the vibrating device 132 generates a vibrating signal (such as a vibrating signal with a frequency of 1 KHZ or 1.5 KHZ). The earhook anti-snoring device 12 of the disclosure disturbs the snoring user through the aforementioned sound signal and vibrating signal so as to achieve the effect of snoring stopping by urging the user to move or change his sleeping posture.

The purpose of this method is that when the user in very deep sleep and the purpose of snoring stopping cannot be achieved by disturbing the user merely by means of a sound signal or vibrating signal of a single frequency (intensity), the possibility of achieving the effect of snoring stopping through disturbance to the user may be improved by changing the frequency (intensity) of the sound signal or the vibrating signal.

The control unit 126 of the earhook anti-snoring device 12 of the disclosure includes a dynamic sensor, such as an acceleration sensor, a gravity sensor or a gyroscope. The dynamic sensor is used for detecting a user's sleep state. That is, the dynamic sensor judges whether the user is in a state of deep sleep, ordinary sleep or wake-up through the times of motions performed by the user. By utilizing the sleep state detected by the acceleration sensor, the earhook anti-snoring device 12 may judge whether the user is in a snoring state more accurately,

After the user continuously uses the earhook anti-snoring device 12 for a period of time, the battery 130 in the earhook anti-snoring device 12 runs out, and at the time the earhook anti-snoring device 12 needs to be charged through the charging base 11. Also referring to FIG. 6, it illustrates a schematic view of a charging base 11 according to an embodiment of disclosure. The charging base 11 includes a spring needle connector 111 and a magnet 112, wherein the spring needle connector 111 is provided with two spring needles, and the magnet 112 is arranged at the position between the two spring needles close to the spring needle connector 111.

The spring needle connector 111 of the charging base 11 is electrically connected with spring contacts of the earhook anti-snoring device 12. When the user places the earhook anti-snoring device 12 above the charging base 11, the magnet 112 of the charging base 11 may have effects of magnetic heteropole attraction (for example, the magnet 112 serves as an S pole and the magnet 134 serves as an N pole) with the magnet 134 of the earhook anti-snoring device 12. By means of the effect of magnetic force, the user may place the earhook anti-snoring device 12 above the charging base 11 more accurately so that the spring needle connector 111 is jointed to the spring connector 133 smoothly, and the battery of the earhook anti-snoring device 12 begins to be charged.

The examples cited above are only optimal embodiments of the present invention, and the features of the disclosure are not limited to this. The variations or modifications, which may be thought of easily in the field of the disclosure by those skills in the art, may fall within the scope of the following claims of the disclosure.

What is claimed is:

1. An earhook anti-snoring device, comprising:
a casing;
a base plate arranged in the casing, wherein the base plate is provided with a first side and a second side;
a control unit arranged on the first side;
a sound-receiving device arranged at one end of the first side, wherein the position of the sound-receiving device corresponds to a first hollow pipe arranged on the casing;
a sound-generating device positioned at one end of the second side and opposite to the sound-receiving device, wherein the position of the sound-generating device corresponds to a second hollow pipe arranged on the casing, and the sound-generating device is directed to an ear canal of a user along the second hollow pipe; and
a vibrating device arranged on the second side.

2. The earhook anti-snoring device of claim 1, further comprising:
a spring connector arranged on the second side, wherein the spring connector is provided with a first spring contact and a second spring contact; and
a first magnet arranged on the second side, wherein the first magnet is adjacent to the spring connector and is located adjacently between the first spring contact and the second spring contact.

3. The earhook ant-snoring device of claim 1, further comprising:
a button;
a switch unit, wherein one end of the switch unit is adjacent to the button; and
a battery arranged at the central position of the first side and opposite to the vibrating device, wherein the battery is electrically connected with the base plate through a line.

4. The earhook anti-snoring device of claim 1, wherein the casing comprises:
a top casing covering the upper portion of the base plate, wherein the top casing is provided with the first hollow pipe, such that the sound-receiving device is maintained open to the outside of the casing along the first hollow pipe; and
a bottom casing covering the lower portion of the base plate, wherein the bottom casing is provided with the second hollow pipe, such that the sound-generating device is maintained open to the ear canal of the user through the second hollow pipe.

5. The earhook anti-snoring device of claim 1, further comprising:
an earplug; and
a ear hook connected with the earplug and the casing.

6. An anti-snoring system, comprising:
an earhook anti-snoring device; and
a charging base, comprising:
a spring needle connector provided with a first spring needle and a second spring needle; and
a second magnet adjacent to the spring needle connector and located adjacently between the first spring needle and the second spring needle.

7. The anti-snoring system of claim 6, wherein the earhook anti-snoring device comprises:
a casing;
a base plate arranged in the casing, wherein the base plate is provided with a first side and a second side;
a sound-receiving device arranged at one end of the first side, wherein the position of the sound-receiving device corresponds to a first hollow pipe arranged on the casing;
a sound-generating device positioned at one end of the second side and apposite to the sound-receiving device, wherein the position of the sound-generating device corresponds to a second hollow pipe arranged on the casing, and the sound-generating device is directed to an ear canal of a user along the second hollow pipe; and
a vibrating device arranged on the second side.

8. The anti-snoring system of claim 7, wherein the earhook anti-snoring device further comprises:
a spring connector arranged on the second side, wherein the spring connector is provided with a first spring contact and a second spring contact; and
a first magnet arranged on the second side, wherein the first magnet is adjacent to the spring connector and is located adjacently between the first spring contact and the second spring contact.

9. The anti-snoring system of claim 7, wherein the earhook anti-snoring device further comprises:
a button;
a switch unit, wherein one end of the switch unit is adjacent o the button; and
a battery arranged at the central position of the first side and opposite to the vibrating device, wherein the battery is electrically connected with the base plate through a line.

10. The anti-snoring system of claim 7, wherein the casing comprises:
a top casing covering the upper portion of the base plate, wherein the top casing is provided with the first hollow pipe, such that the sound-receiving device is maintained open to the outside of the casing along the first hollow pipe; and
a bottom casing covering the lower portion of the base plate, wherein the bottom casing is provided with the second hollow pipe, such that the sound-generating device is maintained open to the ear canal of the user through the second hollow pipe.
